(19) United States
(12) Patent Application Publication
(10) Pub. No.: US 2018/0002281 A1
(43) Pub. Date: Jan. 4, 2018

(54) PRODUCT, OR ACTIVE AGENT, OR COMPOSITION FOR THE CARE OF THE BREASTS IN A PREMENSTRUAL OR MENSTRUAL PERIOD OR FOR THE CARE OF THE SYMPTOMATOLOGY OF MASTODYNIA

(71) Applicants: Stephane DESJONQUERES, MAISONS-LAFFITTE (FR); LABORATOIRES CARILENE, MONTESSON (FR)

(72) Inventor: Stéphane DESJONQUERES, MAISONS-LAFFITTE (FR)

(21) Appl. No.: 15/631,188

(22) Filed: Jun. 23, 2017

(30) Foreign Application Priority Data
Jun. 29, 2016 (FR) 1656119

(51) Int. Cl. C07C 409/04; A61K 31/327; A61K 31/4166; C08G 75/24; C11C 3/00; C11C 3/06

(52) U.S. Cl. CPC C07C 409/04; C08G 75/24; C11C 3/006; A61K 31/327; A61K 31/4166; C11C 3/06

(57) ABSTRACT

A method for caring for or treating the breasts during premenstrual or menstrual phases and for the care or treatment of the symptomatology of mastodynia, comprising a step of selecting or identifying women needing care or treatment of the breasts, or suffering from symptomatology of mastodynia, and a step of applying to the skin areas in need thereof an effective amount of a product, active agent or composition, comprising peroxidized lipids or a peroxidised oil, or consisting essentially of peroxidized lipids or of a peroxidised oil.

The invention relates to a product, or active agent, consisting essentially of, or comprising, peroxidized lipids, for example peroxidized glycerol triesters (abbreviated to TGO), for the care or treatment of the breasts during premenstrual or menstrual phases and also for the care or treatment of the symptomatology of mastodynia. More particularly, the invention relates to a product or active agent consisting of a peroxidized vegetable oil, or comprising a peroxidized vegetable oil, for the care or treatment of the breasts during premenstrual or menstrual phases and also for the care or treatment of the symptomatology of mastodynia.

The invention also relates to a composition comprising a product, or active agent, consisting essentially of, or comprising, peroxidized lipids, for example peroxidized glycerol triesters (abbreviated to TGO), in particular in the form of a peroxidized vegetable oil, for the care or treatment of the breasts during premenstrual or menstrual phases and also for the care or treatment of the symptomatology of mastodynia.

The invention further relates to a method for caring for or treating the breasts during premenstrual or menstrual phases and also mastodynia, comprising a step of selecting women needing care or treatment of the breasts during these premenstrual or menstrual phases or suffering from the symptomatology of mastodynia, and a step of applying an effective amount of a product, or active agent, consisting essentially of, or comprising, peroxidized lipids, for example peroxidized glycerol triesters (abbreviated to TGO), in particular in the form of a peroxidized vegetable oil, for the care or treatment of the breasts during premenstrual or menstrual phases and also for the care of the symptomatology of mastodynia.

### PRIOR ART

It is known that many women have contracted, hard or painful breasts during premenstrual or menstrual phases which are unpleasant and uncomfortable for them and cause them more or less pronounced suffering, the most pronounced stage being described as mastodynia. This state necessarily comprises a hormonal component since it is during the premenstrual and menstrual phases that these phenomena take place.

Thus far, there is no effective product for caring for or treating the breasts during these premenstrual or menstrual phases and even less the most severe state of mastodynia. A single product of hormonal type is marketed under the name Progestogel® and it provides relief considered to be effective.

### PURPOSES OF THE INVENTION

The main purpose of the invention is to solve the technical problem of the provision of a novel solution for the care or treatment of the breasts during premenstrual or menstrual phases and also including the states of the symptomatology of mastodynia.

The main purpose of the invention is also to solve the technical problem of the provision of a novel solution which does not use a hormonal component in order not to interact with the hormonal cycle and often with ongoing contraceptive treatment.

The main purpose of the invention is also to solve the technical problem of the provision of a novel solution which is simple, preferably of plant origin, and which allows the implementation of an industrial production process compatible with the obtaining of a product or of a composition of medical quality with flexibility in terms of the addition of other active ingredients or of other excipients.

The invention simultaneously solves for the first time these technical problems according to a solution that is simple, effective and reproducible on an industrial and medical or para-medical scale.

### DESCRIPTION OF THE INVENTION

According to a first aspect, the invention relates to a product, or active agent, consisting essentially of peroxidized lipids, for example peroxidized glycerol triesters (abbreviated to TGO), or comprising peroxided lipids, for example peroxidized glycerol triesters (abbreviated to TGO), for the care or treatment of the breasts during premenstrual or menstrual phases and also for the care or treatment of the symptomatology of mastodynia.

According to a second aspect, the invention also relates to a composition comprising a product or active agent consisting essentially of peroxidized lipids, for example peroxidized glycerol triesters (abbreviated to TGO), or comprising peroxidized lipids, for example peroxidized glycerol triesters (abbreviated to TGO), for the care or treatment of the breasts during premenstrual or menstrual phases and also the care or treatment of the symptomatology of mastodynia.

According to a third aspect, the invention relates to a method for caring for or treating the breasts during premenstrual or menstrual phases and also for the care or treatment of the symptomatology of mastodynia, comprising a step of selecting or identifying women needing care or treatment of the breasts, or suffering from symptomatology of mastodynia, and a step of applying an effective amount of a product or active agent consisting essentially of peroxidized lipids, for example peroxidized glycerol triesters (abbreviated to TGO), or comprising peroxidized lipids, for example peroxidized glycerol triesters (abbreviated to TGO).

According to a particular feature, for each aspect, the product, active agent or composition is formulated for topical application to the skin of breast areas in need thereof.

Lipids, for example glycerol triesters, constitute the main components of vegetable oils. In the context of the invention, these lipids or vegetable oils containing them are subjected to a controlled peroxidation process in order to obtain peroxidized lipids in particular in the form of a peroxidized vegetable oil.

The peroxidized lipids according to the present invention constitute a film-forming protective agent.

For each of the above aspects, the vegetable oil intended to be peroxidized can be obtained from a vegetable oil chosen in a non-limiting manner from a corn vegetable oil, a soya vegetable oil, a sweet almond vegetable oil, a hazelnut vegetable oil, a peanut vegetable oil, a grapeseed vegetable oil, a sesame vegetable oil and a safflower vegetable oil. Use may also naturally be made of a mixture of these oils in any proportion(s).

According to one particular embodiment of the invention, the peroxidized lipids, which are used in the present invention, result from the peroxidation of lipids or fatty substances of natural origin, in particular of vegetable origin, and, according to one particular variant, of lipids from a natural vegetable oil.

The peroxidized lipids, the use of which is the subject of the present invention, result from the peroxidation of unsaturated fatty substances, which are present in lipids or fatty substances of natural origin and in particular in a natural vegetable oil.

The process for peroxidation of these unsaturated lipids is well known to those skilled in the art. This peroxidation or hyperoxygenation process is carried out by oxygen saturation of the unsaturated lipids, or of the natural form of the unsaturated vegetable oil, with intensive and controlled exposure to ultraviolet rays so as to provide a given degree of peroxidation.

The degree of peroxidation is easily measured by a person skilled in the art according to the ISO 3960 standard method.

In the context of the present invention, peroxidized lipids which have a degree of peroxidation of between 5 and 600 milliequivalents per kg, in particular between 30 and 500 milliequivalents per kg, will be selected for the preparation of the product or active agent or the composition according to the present invention.

The peroxidized lipids used in the context of the present invention comprise, as main constituent, esters of triglycerides generally representing at least 80% of the weight of these lipids, having the formula:

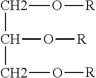

in which the R radicals are mainly represented by partially peroxidized unsaturated C₁₈ fatty acids, depending on the degree of peroxidation of the lipid.

According to one particular variant of implementation of the invention, the product or active agent or the composition, consists essentially of abovementioned peroxidized lipids.

According to another particular variant of the invention, the product or active agent, or the composition, comprises a mixture of the abovementioned peroxidized lipids in particular in a proportion of from 10% to 70% by weight, with another active agent or an excipient compatible with topical administration to the skin, or a mixture of the two, or of several active agents or of several excipients, so as to constitute the remainder of the product or of the active agent or of the composition.

According to another particular variant of the invention, the product or active agent or composition according to the present invention is for topical application to the skin, comprising, as relative percentage by weight:
from 10% to 70% by weight, in particular from 30% to 60% by weight, of peroxidized lipids;
from 0.1% to 10% by weight of an active agent or excipient with thickening function, for adjusting the viscosity;
from 0.1% to 10% by weight of an active agent or excipient of which a function is to solubilize the peroxidized lipids or the peroxidized oil;
optionally from 0.1% to 5% by weight of a broad-spectrum antimicrobial agent.

According to a further particular variant of implementation of the invention, the product or the composition is characterized in that it comprises approximately 50% by weight of peroxidized lipids.

According to another particular variant of implementation of the invention, the active agent or excipient having a thickening function may comprise a polymer of sulphonic acids, in particular in the form of an ammonium salt, and even more particularly ammonium acryloyldimethyl taurate/VP copolymer. Substances of this type are well known to those skilled in the art and are for example sold under the brand name Aristoflex AVC 0025 by the company Clariant Productions, at 94 Choisy Le Roi, France.

According to one particular variant of the invention, the active agent or excipient of which the function is to solubilize the peroxidized lipids or peroxidized oil can comprise a non-ionic surfactant, for example a hydrogenated castor oil, for example modified with polyethylene glycol, alone or combined with another non-ionic surfactant substituted with polypropylene glycol. This is thus a synergistic mixture of non-ionic surfactants.

A mixture of these two substances is well known to those skilled in the art and is commercially available under the name Solubilisant LRI®, sold by the company Sensient Cosmetic Technologies, 95310 Saint Ouen I'Aumône. This solubilizing product is generally in the form of a viscous liquid and is also used to adjust the viscosity of the product or of the composition according to the present invention.

According to another particular variant of implementation of the present invention, an antimicrobial active agent is generally present. Said active agent may comprise one or more active ingredients.

One particular embodiment of the invention comprises a use as antimicrobial agent of a mixture of 2-phenoxyethanol and of 1,2-propanediol, and 3-(4-chlorophenoxy) or chlorphenesin, and also glycerin.

This product is commercially available under the name Microcare® PHC, sold by the company Thor Personal Care SAS, 90 La Croix Saint Ouen.

According to another particular embodiment of the invention, the product or active agent or the composition also comprises allantoin. It has been observed, unexpectedly, that the combination of allantoin with the peroxidized lipids contributes to the cosmetic qualities of the final product, in particular with regard to the rancid odour characteristic of peroxidized oils.

Indeed, it has been discovered, surprisingly, that allantoin fixes and stabilizes corrective fragrances in order to in the end exploit the olfactory qualities of the final product.

According to one particular implementation variant, the proportion of allantoin can generally be between 0.1% and 2% by weight, in particular between 0.5% and 1.5% by weight.

The breast symptoms during premenstrual or menstrual phases, or in the case of the symptomatology of mastodynia, comprise a tension, heaviness of the breasts, a hot sensation, in certain cases an inflammation, the existence of pain, in certain cases extreme pain, and oedema.

The product, or active agent, or the composition, according to the invention makes it possible to relieve all these symptoms.

The product, or active agent, or the composition, according to the present invention can be in various galenical forms.

For example, the product, or active agent, or the composition, according to the invention can be in the form of a cream which, according to one particular embodiment, can comprise:
oxy-unsaturated fatty acid triglycerides according to the present invention in a proportion by weight of from 30% to 70%, in particular between 40% and 60% by weight and more particularly at approximately 50% by weight.

The rest of the composition can comprise various other active agents or excipients in an amount sufficient to make the remainder up to 100% by weight.

The active agents or excipients have been indicated above, obviously in a non-limiting manner.

Other purposes, features and advantages of the invention will emerge clearly from the following description of several compositions of the invention, given simply by way of illustration and which could not in any way limit the scope of the invention, and also a clinical study.

The examples are an integral part of the invention and include all the technical equivalents. In the examples, all the percentages are given by weight, the temperature is ambient temperature or is in degrees Celsius, unless otherwise indicated.

### EXAMPLES OF THE INVENTION AND CLINICAL STUDY

### Example 1 of the Invention

Product or composition of the present invention in the form of a cream comprising:
a) peroxidized triesters of glycerol or else oxy-unsaturated fatty acid triglycerides having a degree of peroxidation of between 5 and 600 milliequivalents per kg, at a proportion of approximately 50% by weight,
b) active agents or excipients comprising:
b1) a solubilizing excipient, also constituting a mixing or emulsifying agent, for example the commercially available solubilizer LRI® in a proportion of approximately 2% by weight,
b2) an antimicrobial preservative, for example the commercial product Microcare® PHC, in a proportion of approximately 1% by weight, and
b3) cosmetic-quality or pharmaceutical-quality demineralized water, as solvent for producing the emulsion, QS 100%.

This emulsion is prepared conventionally.

Herein, the peroxidized glycerol triesters, for example obtained from peroxidized corn oil, will first of all be mixed with the mixing or emulsifying solubilizing agent LRI®, then the antimicrobial preservative Microcare® PHC, and the water will finally be added while mixing vigorously so as to form an emulsion.

The mixture can be prepared at ambient temperature or by heating as is conventional in the case of the production of emulsions.

### Example 2 of the Invention

Topical Product or Composition for Clinical Study

This topical product or composition for clinical study comprises the following composition:

| | |
|---|---|
| a) peroxidized glycerol triesters obtained from peroxidized corn oil, | approx. 50% |
| having a degree of peroxidation of between 5 and 600 milliequivalents | |
| per kg | |
| b) active agents or excipients, comprising | |
| b1) an emulsifying and solubilizing mixing agent, | approx. 2% |
| for example Solubilisant LRI ® | |
| b2) antimicrobial agent | approx. 1% |
| for example Microcare ® PHC | |
| b3) texturing or thickening agent, | approx. 1% |
| for example Aristoflex ® AVC | |
| b4) optionally a fragrance or a mixture of | approx. 1% |
| fragrances for improving the odour | |
| for example: | |
| cornflower-based fragrance 0.50% | |
| white lotus fragrance 0.40% | |
| b5) cosmetic-quality or pharmaceutical-quality demineralized water, | |
| as solvent for producing the emulsion, QS 100%. | |

This emulsion is prepared conventionally, as indicated in example 1.

The mixing of the ingredients is carried out as in the context of example 1 in order to obtain a composition in the form of an emulsion having the consistency of a cream, that is called RD6.

This composition is the subject of the following clinical study reported in example 4.

### Example 3 of the Invention

Topical Product or Composition Comprising Allantoin

This topical product or composition comprising allantoin, for clinical study, comprises the following composition, as % by weight:

| | |
|---|---|
| a) peroxidized glycerol triesters obtained from peroxidized corn oil | approx. 50% |
| having a degree of peroxidation of between 5 and 600 milliequivalents | |
| per kg | |
| b) allantoin excipient | approx. 0.5% |
| c) other agents or excipients | |
| c1) emulsifying and solubilizing mixing agent, | approx. 2% |
| for example Solubilisant LRI ® | |
| c2) antimicrobial agent, | approx. 1% |
| for example Microcare ® PHC | |
| c3) texturing or thickening agent, | approx. 1% |
| for example Aristoflex ® AVC | |
| c4) optionally a fragrance or a mixture of | approx. 1% |
| fragrances for improving the odour | |
| for example: | |
| cornflower-based fragrance 0.50% | |
| white lotus fragrance 0.40% | |
| c5) cosmetic-quality or pharmaceutical-quality demineralized water, | |
| as solvent for producing the emulsion, QS 100%. | |

This composition using allantoin as excipient fixes and stabilizes the corrective fragrances in order to in the end exploit the olfactory qualities of the final product.

### Example 4 of the Invention

Clinical Study Carried Out with the Product or Composition of Example 2

I) Materials and Methods

a) The study comprised the inclusion of 45 women, 21 in the context of senology consultation in a hospital or in a private clinic and 24 among the patients of an independent practice.

The inclusion criteria were the following:
female subjects whose ethnicities were of no importance;
aged from 20 to 50;
accepting to participate in the study after signing a consent;
presenting premenstrual signs of pain and congestion in the mammary gland.

The exclusion criteria were the following:
women with a progressive organic pathological condition;
pregnant women;
women having had a recent or carcinology-related history of mammary surgery;
women exhibiting a known intolerance or hypersensitivity to derivatives of fatty acid triglycerides of vegetable origin.

During the study, there were no lifestyle modifications, nor any change in the medical treatment ongoing on the day of consultation, treatment given by questioning the patient.

b) The Method

This clinical study was carried out over a period corresponding optimally to 3 menstrual cycles of the woman.

Prior to her inclusion, the patient received from the physician information concerning the product studied, the purposes, the methods and the expected benefits.

After having given this oral and written information, the physician collected two copies of the patient's written consent, one for the patient and the other kept by the physician.

c) Implementation of the Study

The initial assessment was carried out during the first medical visit and comprised:
a diagnosis of tension, pain and heaviness in the breasts associated with premenstrual syndrome with assessment by the physician and by the patient on analogical scales. These scales were graded from 0 to 10: 0 corresponding to the total absence of a sign and 10 corresponding to an intolerable presence of this sign;
an objective evaluation of these signs carried out by clinical examination of the patient by the physician.

This first visit was that of the inclusion day (C0).

The second visit took place during the premenstrual period, approximately one week before the presumed date of the next period. This visit, called C1, allowed a first evaluation of the treatment after the use of the product of example 2 over the course of one cycle.

The third visit, called C2, was set at approximately one week from the presumed date of the period of the 4th cycle, that is to say in total between the 3rd and the 4th month after inclusion.

This visit allowed the evaluation of the treatment after the use of the product of example 2 for three consecutive cycles.

The analysis of the results of the study made it possible to compare the strength of the symptoms, and also the rapidity of improvement of the discomfort assessed by the patient and the durability of this improvement, and also the improvement in the phenomena that may be inflammatory and also oedema before and after treatment.

II—Results of the Study

A) Population

The study related to 45 included patients. 34 patients were able to have a complete study (75.6%); the evaluation of the product of example 2 was carried out on all of these 34 patients.

5 patients left the study prematurely, that is to say 11.1%: one because of a local allergy in a patient with a marked allergic history; one because of a nervous breakdown; two who, having observed no improvement after the first month, wanted to stop; they presented clinically a very intense mammary dystrophy and one wanted to stop the study because of intolerance to the odour and to the excessively fluid texture.

4 patients did not return, that is to say 8.9%, despite several requests to return by the secretarial offices.

The patients included were between 20 and 49 years old, on average 36.4 years old, with uniformity in the two populations.

With regard to how long the symptomatology of mastodynia had been going on, it ranges from 1 to 264 months with an average of 44.2 months. The period of time appeared to be slightly longer in the independent practice compared with the hospital.

B) Duration of the Study

The duration of the study was on average 99.5 days (with extremes of 50 to 213 days). The study was longer in the independent practice because of the lesser availability of the patients, 113.80 days in the independent practice compared with 88.16 days in the hospital environment. When analysing the duration of the study more finely, it should be noted that 4 patients, 2 in each population, greatly exceeded the deadline set for the time between the first consultation and the second consultation.

With regard to the period of time between the second consultation (C1) and the third consultation (C2), in 10 cases, the patients greatly exceeded the time period proposed in the study.

All ten abovementioned cases involved a month of vacation for the physician and the patients, 7 in the independent practice and 3 in the hospital environment.

Moreover, 11 patients had a much shorter period of time, one in the independent practice and 10 in the hospital environment.

Finally, with regard to the total duration of the study between the first inclusion visit and the third visit, a period of time greater than what was foreseeable for 7 patients, 5 in the independent practice and 2 in the hospital environment, and 9 patients whose period of time was much shorter, 3 in the independent practice and 6 in the hospital environment, are noted.

C) Associated Symptoms

Said symptoms were found to a larger extent in the hospital population compared with the patients of the independent practice, 17 patients in the hospital environment compared with 7 patients in the independent practice, given that the symptoms were the following:
7 times a bilateral brownish or greenish, or even lactescent, discharge, and
2 times an old dystrophy, and
once a history of adenofibroma ablation, and
once an old fibrocystic disease, and
in 6 cases, particularly intense mastodynia with, for one patient, the taking of oral contraception.

In the independent practice, the following are found:
1 dystrophy,
3 times mastodynia while taking oral contraception,
1 patient presenting premenstrual migraine,
2 patients presenting problems with periods, algomenorrhoea or metrorrhagia, these 2 patients being, moreover, treated with macro-progestogens.

D) Analogical Scales

These scales enabled the 34 patients to evaluate the subjective signs during the three consultations, C0, C1 and C2.

These assessment criteria related to the pain, tension and heaviness in the breasts.

1) On inclusion (C0) on average
the pain was graded 5.99 with extremes of 0 to 9.36, 6.15 for the hospital patients, 5.80 for the independent practice patients;
the breast tension was graded 5.77 with extremes ranging from 0 to 9.03. A clear difference between the grading of the hospital patients at 7.22 and those of the independent practice patients at 3.95 is noted;
the heaviness was graded at 5.6 with extremes ranging from 0 to 10 and, here again, a difference is found with a grading at 6.83 in the hospital environment compared with 4.14 in the independent practice.

2) During the second consultation (C1), that is to say at least after one complete treatment cycle, these various parameters were re-evaluated:
the pain was graded on average at 3.14 with extremes of 0 to 9.84, a difference between the hospital patients at 3.82 and the independent practice patients at 2.28.

The improvement compared with the first consultation was 48%: 37% for the hospital patients and 60% for the independent practice patients;
the breast tension was graded on average at 3.21 with extremes of 0 to 7.26: 3.89 for the hospital patients compared with 2.35 for the independent practice patients.

Here again, the improvement compared with the first consultation was 45%: 46% for the hospital patients and 41% for the independent practice patients;
the heaviness: graded on average at 2.90 with extremes of 0 to 8.22: 3.67 for the hospital patients and 1.92 for the independent practice patients with an average improvement of 48%: 74% for the hospital patients and 53% for the independent practice patients.

3) During the third consultation (C2) optimally carried out after three treatment cycles;
the pain was graded on average at 2.19 with extremes of 0 to 8.75: 1.96 for the hospital patients compared with 2.48 for the independent practice patients.

The improvement in the patients had progressed between the second consultation (C1) and the third consultation (C2) at 29%, that is to say 50% in the hospital context, but no improvement was noted in the independent practice context;
the tension was graded 1.68 with extremes ranging from 0 to 5: 1.56 for the hospital environment compared with 1.84 for the independent practice.

Here again, the improvement was 47% compared with the (C1) consultations, 59% in the hospital environment compared with 21% in the independent practice;
the heaviness was graded at 1.75 on average with extremes ranging from 0 to 5.28: 1.67 for the hospital environment compared with 1.84 for the independent practice.

The improvement between C1 and C2 was found to be 38%: 54% for the hospital patients compared with 5% for the independent practice patients. In total, between the first inclusion consultation (C0) and the final consultation (C2) corresponding to an optimal treatment of 3 cycles, there is a notable improvement:
for the pain in 63% of cases, 68% in the hospital environment and 57% in the independent practice,
for the tension in 71% of cases, 79% in the hospital environment and 53% in the independent practice,
for the heaviness in 69% of cases, 76% in the hospital environment and 56% in the independent practice.

E) Objective Clinical Evaluation

1) Weight

There was no significant variation in the patients during the study:
at C0=57.4 kg (with extremes ranging from 49 to 71 kg)
at C1=57.3 kg
at C2=57.2 kg

Between C0 and C2, an average difference of 0.247 g is noted, with extremes of −3.5 kg and of +5 kg, corresponding, for the 2 patients, to vacation times.

Only 10 patients experienced a decrease in their weight during the study, that is to say 10 out of 34=29.4%
0.5 kg for 2 patients
1 kg for 6 patients
3.5 kg for 2 patients.

2) Bust Measurement

Of all of the patients, an average bust measurement of 88 cm was found (with extremes of 80 to 101 cm) with
at C0=88.5 cm
at C1=88.6 cm
at C2=88.3 cm

The average difference between C0 and C1 is −0.1 and between C0 and C2 it is −0.1.

The greatest differences are between C0 and C1 of +4 cm in 2 patients and −3.5 cm between C0 and C2 of +4 cm and −3.5 cm (same patients as previously).

3) Bra Size

5 patients exhibited a notable variation in their bra size:
for one of them, a slight increase in cup size, 85B to 85C at C2;
for the other 4, a decrease:

| | C0 | C1 | C2 |
|---|---|---|---|
| Patient No. 1 | 90B going to 90D | 90D | 90C |
| | in the | | |
| | premenstrual | | |
| | period | | |
| Patient No. 2 | 85C | 85B | 85B |
| Patient No. 3 | 90B | 90B | 90A |
| Patient No. 4 | 90B | 90D | 90D |

For patient No. 1, all of the functional signs rapidly disappeared as early as C1
pain 6.13 [symbol] 0
tension 6.94 [symbol] 0
heaviness 9.36 [symbol] 0

with virtually immediate relief, one hour after application.

4) Skin Temperature

The measurements extend between N (“normal” temperature or temperature below 37°), 37° and 38°. The most common differences are by one unit. In nine patients, a difference of 2° (from 38 [symbol] 37-38 [symbol] N) is noted; seven of these nine patients are from the hospital group and three of them present an organic pathological condition of galactophorous discharge type.

5) Clinical Examination

5.1—Breast Tension
at C0: 76.47% of women had mammary tension
at C1: 58.82% of the patients presented this clinical sign.

The following are thus noted:
an absence of improvement in 7.14% of cases
a noticeable improvement in 39.29% of cases
a clear improvement in 39.29% of cases.

This corresponds to an overall improvement in 22 patients out of 28 presenting this symptom:
at C2: 51.52% of patients presented persistent mammary tension.

In total, between C0 and C2, the following are noted:
an absence of improvement in 3.57% of cases
a notable improvement in 37.71% of cases
a clear improvement in 50% of cases.

This makes it possible to conclude that there is a satisfactory improvement in 24 out of 28 patients.

5.2—Granular Consistency on Examination
during the inclusion, this clinical aspect was found in all of the patients,
during the first consultation, this aspect was found in 94.12% of the patients,
at C2, it was found in 79.41% of the patients.

This demonstrates, between C0 and C1:
a notable improvement in 44.12% of patients,
a clear improvement in 29.41% of patients.

The set of patients showing improvement is 25 out of 34 (73.5%).

Between C1 and C2, the following are noted:
the notable improvement was 37.50%
the overall improvement was found in 18 patients out of 34 (53%).

This makes it possible to conclude that there is a satisfactory improvement in 24 patients.

In total, between C0 and C2, the following are noted:
the absence of result in 8.82% of patients
a notable improvement in 23.53% of patients
a clear improvement in 64.71% of patients.

This corresponds to an overall improvement for 30 patients out of 34.

5.3—Nipple Discharge
during the inclusion, 10 patients had this clinical sign for 2 patients
at C1: 9 patients
at C2: 8 patients.

The improvement was
complete with disappearance of the clinical sign for 2 patients
zero for 4 patients
graded moderate for 4 patients with less abundant discharge.

5.4—Existence of a Network of Veins

This sign was frequently found during inclusion in 24% of patients (67.65% of cases).

Between C0 and C1, the following are noted:
the absence of improvement in 54.16% of cases
a notable (47.67%) or clear (12.5%) improvement in 19 patients out of 24.

5.5—Oedema

This sign was evaluated by measuring the skin fold:
at inclusion, 18 patients out of 34 presented this symptom
at C1, it persisted in 13 patients (38.24%)
at C2 in 6 patients (17.66%).

Between C0 and C1, the following are noted:
a good improvement in 66.67% of cases
a very good improvement in 27.77% of cases.

This gives an overall improvement in 17 patients out of 18.

In total, between C0 and C2, there is a 100% improvement with good results in 33% of patients and very good results in 67% of patients.

5.6—the Presence of Axillary Lymph Nodes

The presence of axillary lymph nodes is a reflection of the state of breast inflammation.

This sign was found in 13 patients, which corresponds to 38.24% of patients;
at C1: 9 patients still presented axillary adenopathies
at C2: 7 (20.59%) this makes it possible to find an improvement in this symptom in 4 patients out of 12.

In total, the improvement between C0 and C2 was noted in 8 patients tested out of 13.

6) Cosmetic Evaluation of the Product

The analysis of the assessment, by the patients, of the cosmetological qualities of the product of example 2 is quite well-matched since:
the texture is very pleasant or pleasant for 23 patients with a feeling of rapidly obtained freshness and of softness of the skin after penetration of the product;
the ease of application is well-liked for 93.93% of patients.

The only restrictions concern:
the odour described as “fries” or “butter” by 54% of the women,
the presentation form in a jar (tendency to leak when subjected to heat).

In total: with the proviso of a modification of the presentation form (measuring bottle for example) and of the odour, the product of example 2 remains a product that is very well received for its fast-penetrating fluid texture, and the simple and fast, non-restrictive and efficient application: 84.85% of patients are ready to continue this treatment.

III) Discussion

This study of the product of the invention relates to 34 patients. Very few patients left the trial for reasons associated with the product: one allergy, but the patient had a known history, one for intolerance to the odour although it was effective and in reality only two (2/34: 5.8%) for lack of effectiveness.

A better adherence to the time period of the various consultations is observed in hospital because of the recruitment which brought into the study mostly the medical or paramedical female population of the hospital, outside the particular time of vacations where extremes could be observed.

How long the problems have been ongoing is a piece of information that is not very reliable since it is a notion based solely on the patients' recollection.

All of the patients consulting in the hospital present more frequently an associated pathological condition; this is directly linked to the very surgical recruitment of the hospital consultation and to the longer period of time of the consultation itself, selecting in this way the patients who present more pronounced pathological conditions with the presence of associated organic signs.

At the time of the inclusion visit, this is very clearly demonstrated on the analogical scales of breast tension (7.22 compared with 3.95) and of heaviness (6.83 compared with 4.14).

From the first month of treatment, the decrease in pain is better and faster in the independent practice (60% improvement compared with 37%).

This demonstrates the speed with which the product is effective on pain; this speed can be linked to better adherence by the patients of the independent practice with a more regular application, but it is also this population where it is noted that, at inclusion, the strength of the pain for symptomatology is more moderate.

The improvement in the signs appears to be clearer during the third consultation (C2) of the C.H.U. (University Hospital Center) patients.

This thus confirms:
the maximum effectiveness from the first month on the symptomatology of mastodynia associated with oedema,
the need for a more prolonged treatment in patients with an underlying organic pathological condition.

With regard to weight, no significant difference was found during the study apart from for one patient, but this coincides with a change in lifestyle (vacation period). This piece of data could only be varied in the case of considerable premenstrual syndrome with intense breast oedema, which was not found in the study.

The variations in bust measurement are overall not very significant in terms of average difference. Furthermore, it is a measurement subject to variability from one examination to the other for one and the same examiner. In the patient who experienced a decrease in her bust size of 3.5 cm, this observation is made as early as C1 and found at C2. This is a woman in whom the treatment was particularly effective:
on pain graded 9.03 at C0 and 0 at C1;
on tension graded 6.29 at C0 and 0 at C1;
on heaviness graded 9.36 at C0 and 0 at C1.

For the patient having put on an additional 4.5 cm, a good decrease in pain is noted during the three of treatment (7.26 at C0 [symbol] 1.29 at C1 [symbol] 1.53 at C2), but the tension remains considerable, or even worsened (1.29 at C0 [symbol] 5.19 at C1 [symbol] 3.33 at C2) as does the heaviness (5.32 at C0 [symbol] 8.22 at C1 [symbol] 4.03 at C2). In fact, this women was seen at C2 ten days before her period and had mastodynia on that day, whereas overall she was rather satisfied with the treatment.

Two patients had a bust measurement graded at +4 cm at C1;
one was seen at C1 without pain or tension but with notable heaviness at 5.97 on the analogical scale, whereas at C2, right in the middle of a pain attack, the bust measurement was identical (pain: 8.20, tension 3.75, heaviness: 7.36);
for the other patient, a paradoxical evolution is noted (C0: 86, C1: 90, C2: 88) since she rapidly obtained relief for:
the pain: 8.55 at C0 [symbol] 2.26 at C1 [symbol] 0.7 at C2;
the tension: 7.58 at C0 [symbol] 2.26 at C1 [symbol] 0.56 at C2;
the heaviness: 7.58 at C0 [symbol] 2.42 at C1 [symbol] 0.56 at C2.

Outside these extreme results, a clear correlation is found between the breast heaviness and tension signs and the increase in the bust measurement; these phenomena are closely linked to oedema and to vascular congestion.

When the functional symptomatology decreases significantly, it is accompanied by a decrease in the objective clinical signs (decrease in the bust measurement size and decrease in bra cup size) through a decrease in congestion and in oedema. When the patients are questioned, the notion of “feeling better”, of “a bra that is not as tight” or “less full” is virtually unanimously found, without the patient feeling the need to buy a new one of smaller size.

The correlation between the skin temperature and the subjective functional signs shows that the 9 patients rapidly as a whole obtain considerable relief between C0 and C2. The improvement was:
from +0.06 to −5.8 pts for the pain
from −3.3 to −7.2 pts for the breast tension
from +1.11 to −8.34 pts for the heaviness.

As the study progresses, an increase in the percentage of the women who no longer present breast tension on clinical examination is observed, since the figures go from 23.53% at the inclusion visit to 48.48% at C2. In order to obtain the maximum improvement (50% of the women), three months of treatment are therefore necessary since, in one month, only 39.29% of very good results are obtained; with regard to the notable improvement, it appears to be less subject to variations, with a maximum as early as the first month of treatment (C0: 39.29% [symbol] C1: 40.91% [symbol] C2: 35.71%).

If the results are examined as a function of the various populations, it appears that the maximum improvement is observed in the hospital population since the maximum tensions coded ++++ are more frequent at C0 with improvements coded slight to +, or even “very supple breasts”, that is to say absence of tension, at C2.

In total: the improvement with regard to tension is all the more remarkable since a pre-existing organic pathological condition appears to exist, that is to say that, here again, the tension phenomena decrease as the vascular congestion and the oedema decrease.
The maximum improvement in the granular aspect is obtained after three months of treatment since the figures go from 29.41% to 64.71% of very improved patients, that is to say that a result entirely parallel with the improvement in the breast tension is observed.
The notable improvement is obtained as early as the first month of treatment in 44.12% of patients, whereas it is then only 23.53% after three months of treatment.

In terms of population, here again, it is especially the hospital population, that is to say the population with a pre-existing pathological condition, which presents maximum results at C0: 17 patients coded +++ or ++++ compared with 5 in the independent practice, whereas at C2, no woman has this type of coding in the hospital population, whereas 3 patients are still coded at +++ in the independent practice population.

The unilateral or bilateral nipple discharge found in 10 patients out of 34 appears to be modified little by the treatment; it seems logical that the oedema and the vascular congestion are not involved in the appearance of this symptom which is clearly linked to a pathological duct condition (ectasia, cystic dystrophy) and not to an interstitial pathological condition.

The improvement with regard to the network of veins appears to be moderate between C0 and C1 since the number of patients who have improved varies little, with a clear improvement in a single patient (4.17%), a moderate improvement in 10 patients, while 13 have no improvement.

On the other hand, after three months of treatment, in 79.17% of patients, a notable or clear improvement is noted, which corresponds to 19 patients out of 24. A treatment for three months is therefore necessary in order to have the maximum effectiveness on the superficial network of veins.

The maximum effectiveness on oedema is obtained after three months of treatment since the very good results go from 27.77%, after one month of treatment, to 66.67% after three months of treatment, but very rapid relief is observed as early as the first month since 17 women out of 18 have a good or very good result.

In terms of population, the maximum variations are observed mainly in the town population between C0 and C2 (+−3 and −4 millimetres on the skin fold, compared with −2.5 millimetres in the hospital population). Since the town population groups together mainly patients with premenstrual gastrodynia, the oedema regresses more notably in a larger number of women.

The presence of axillary adenopathies is found in 12 patients out of 21 at C0 in the hospital population, whereas they are present only in two town patients; this can be explained by the notion that, in the hospital, the women are mainly carrying a pre-existing pathological condition (fibrocystic disease, dystrophy, etc.).

After three months of treatment, 7 women out of 12 of the hospital still present adenopathies, the 2 town patients are free of them.

In total: in the event of a pre-existing pathological condition, adenopathies are frequent and do not disappear very much after three months of treatment.

IV) Conclusion

This study, carried out in Rouen, both on a population of patients consulting in an independent practice and on patients from a senology consultation in the hospital, appears to be particularly interesting from several aspects:
1. the diversity of the clinical symptomatology and of the associated signs of the patients included;
2. the very small number of patients having abandoned the study for reasons inherent in the product itself (texture, absence of improvement);
3. the very rapid and especially unquestionable effectiveness of the product both with regard to the subjective clinical signs and with regard to the clear improvement in the breast examination carried out by the clinician;
4. the virtual unanimity of the patients completely satisfied with the relief provided by the product and their desire to continue the treatment.

1. A method for caring for or treating the breasts during premenstrual or menstrual phases and also for the care or treatment of the symptomatology of mastodynia, comprising a step of selecting or identifying women needing care or treatment of the breasts, or suffering from symptomatology of mastodynia, and a step of applying to the skin areas in need thereof an effective amount of a product, active agent or composition, comprising peroxidized lipids or a peroxidised oil, or consisting essentially of peroxidized lipids or of a peroxidised oil.

2. The method of claim 1, wherein the peroxidised lipids or peroxidised oil comprise/s or are/is consisting of peroxidized glycerol triesters.

3. The method of claim 1, wherein the peroxidised lipids or peroxidised oil are/is obtained from a vegetable oil chosen from a corn vegetable oil, a soya vegetable oil, a sweet almond vegetable oil, a hazelnut vegetable oil, a peanut vegetable oil, a grapeseed vegetable oil, a sesame vegetable oil and a safflower vegetable oil, or a mixture of these oils in any proportion(s).

4. The method of claim 1, wherein the peroxidized lipids/peroxidised oil have/has a degree of peroxidation of between 5 and 600 milliequivalents per kg.

5. The method of claim 1, wherein the peroxidized lipids/peroxidised oil comprise/s triglyceride esters, representing at least 80% of the weight of these lipids, having the formula: 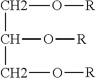
in which the R radicals are represented by partially peroxidized unsaturated C₁₈ fatty acids, depending on the degree of peroxidation of the lipid.

6. The method of claim 1, wherein the product, active agent or composition is consisting essentially of the peroxidized lipids or peroxidised oil.

7. The method of claim 1, wherein the product, active agent or composition comprises a mixture of the peroxidized lipids or peroxidised oil in a proportion of from 10% to 70% by weight with another active agent or an excipient compatible with topical administration to the skin, or a mixture of the two, or of several active agents and of several excipients, in order to constitute the remainder of the product, active agent or composition.

8. The method of claim 1, wherein the product, active agent or composition is for topical application to the skin, and comprises, as relative percentage by weight:
from 10% to 70% by weight, of peroxidized lipids or peroxidised oil;
from 0.1% to 10% by weight of an active agent or excipient with a thickening function for adjusting the viscosity;
from 0.1% to 10% by weight of an active agent or excipient with a solubilizing function for the peroxidized lipids or the peroxidized oil;
optionally from 0.1% to 5% by weight of a broad-spectrum antimicrobial agent.

9. The method of claim 8, wherein the product, active agent or composition comprises from 30% to 60% by weight of peroxidized lipids or peroxidised oil.

10. The method of claim 8, wherein the active agent or excipient having a thickening function is selected from the group consisting of a sulphonic acid polymer, an ammonium salt of a sulphonic acid polymer, and ammonium acryloyldimethyl taurate/VP copolymer.

11. The method of claim 8, wherein the active agent or excipient having a solubilizing function of the peroxidized lipids or peroxidized oil comprises a non-ionic surfactant.

12. The method of claim 11, wherein the non-ionic surfactant comprises a hydrogenated castor oil substituted with polyethylene glycol alone or combined with another non-ionic surfactant substituted with polypropylene glycol.

13. The method of claim 8, wherein the product, active agent or composition comprises from 0.1% to 5% by weight of an antimicrobial active agent.

14. The method of claim 13, wherein the antimicrobial active agent comprises a mixture of 2-phenoxyethanol and of 1,2-propanediol, and 3-(4-chlorophenoxy) or chlorphenesin, and glycerin.

15. The method of claim 1, wherein the product, active agent or composition comprises from 0.1% to 2% by weight of allantoin.

16. The method of claim 8, wherein the product, active agent or composition comprises from 0.5% to 1.5% by weight of allantoin.

17. The method of claim 8, wherein the product, active agent or composition comprises about 50% by weight of peroxidized lipids.

18. A composition for topical application to the skin, consisting essentially of peroxidized lipids or comprising an effective amount of peroxidized lipids for the care or treatment of the breasts during premenstrual or menstrual phases, and for the care or treatment of the symptomatology of mastodynia.

19. The composition of claim 18, comprising a mixture of the peroxidized lipids, with another active agent or an excipient compatible with topical administration to the skin, or a mixture of the two, or of several active agents or of several excipients.

20. The composition of claim 18, comprising from 10% to 70% by weight of the peroxidized lipids.

21. The composition of claim 18, wherein the peroxidized lipids comprise or are essentially consisting of peroxidised glycerol triesters.

22. A composition for topical application to the skin, comprising, as relative percentage by weight:
from 10% to 70% by weight, of peroxidized lipids or peroxidised oil;
from 0.1% to 10% by weight of an active agent or excipient with a thickening function for adjusting the viscosity;
from 0.1% to 10% by weight of an active agent or excipient with a solubilizing function for the peroxidized lipids or the peroxidized oil;
optionally from 0.1% to 5% by weight of a broad-spectrum antimicrobial agent.

23. The composition of claim 18, comprising from 0.1% to 2% by weight of allantoin.

24. The composition of claim 22, comprising from 0.1% to 2% by weight of allantoin
